(19) United States
(12) Patent Application Publication
(10) Pub. No.: US 2023/0352127 A1
(43) Pub. Date: Nov. 2, 2023

(54) Method and System for Automatic Electronic Health Record Documentation

(71) Applicants: Sridharan Sivan, Chennai (IN); Sushindri Sridharan, Chennai (IN)

(72) Inventors: Sridharan Sivan, Chennai (IN); Sushindri Sridharan, Chennai (IN)

(21) Appl. No.: 17/841,633

(22) Filed: Jun. 15, 2022

(30) Foreign Application Priority Data
Apr. 29, 2022 (IN) 202241025148

(51) Int. Cl. G16H 10/60; G06F 40/30; G06F 40/20; G10L 15/26

(52) U.S. Cl. CPC G16H 10/60; G06F 40/30; G06F 40/20; G10L 15/26

(57) ABSTRACT

The present invention relates to a method and a system for automatic electronic health record documentation. It uses knowledge engineering to convert recordings or diarized texts of interactions between a medical practitioner and a patient into a narrative and entering the data into the desired electronic health record. The present invention is trained at the voice level to identify medical concepts spoken in different accent and auto identify the medical context in speech. The present invention learns the electronic health record (EHR) workflow of the medical practitioner and mimics the clinical workflow and protocol to ensure all elements as per the practitioner are in place and logs into the EHR and enter the data into a structured format.

### FIELD OF INVENTION

The present invention generally relates to the field of machine learning; Particularly, the present invention relates to the method and system for automatic electronic health record documentation.

### BACKGROUND OF THE INVENTION

Healthcare service providers have been conventionally keeping all of their patients' information in paper filing systems. That patient information includes, but is not limited to, patients' demographic information (e.g., age, weight, gender, race, income, and geographic location), financial information (e.g., outstanding balances, insurance claims currently being processed, and other account information), and clinical information (e.g., clinician documentation of observations, thoughts and actions, treatments administered, patient history, medication and allergy lists, vaccine administration lists, laboratory reports, X-rays, charts, progress notes, consultation reports, procedure notes, hospital reports, correspondence, and test results).

The healthcare providers, or clinicians, that maintain that patient information include, but are not limited to, physicians (Doctors of Medicine (MDs) and Doctors of Osteopathic Medicine (DOs)), dentists, chiropractors, pediatrists, therapists, psychologists, physician assistants, nurses, medical assistants, and technicians.

The manual, paper-based practice of keeping a patients' information, however, is a very inefficient, labor-intensive process that requires many checks and balances to ensure accurate processing of the information and, therefore, takes up a significant amount of clinician's time that could otherwise be spent with patients. Accordingly, electronic medical records (EMRs), Electronic Health Records (EHRs), and Personal Health Records (PHRs) have been developed to provide many of the functionalities and features of paper filing systems in an electronic, paperless format.

Conventional methods involve Doctors spending between 1.5 hrs to 4 hours on EHR software every working day to create clinical documentation. This leads to over work, loss of productivity and burnout among doctors.

To overcome these problems the physicians have started employing scribes to do the data entry into EHR and electronic medical records documentation. Using scribes has become very costly and could range between $2500 to $3500 per month, patients also feel awkward to have a physical or virtual scribe when the doctor is reviewing the systems. High turnover and low retention of scribe leads to Physicians training the new scribes about his workflow and documentation process over and over again leads to additional stress and work load for the doctors.

Some of Physicians also have tried to use recorded conversation model along with a virtual scribe to create SOAP notes which can be copy pasted to the EMR or in some cases integrated to the EHR software. Since it involves scribes to edit the documents, it has all the challenges of involving a human in the process. This also has challenges of integration which creates its own additional issues.

The exhaustive literature search conducted to identify prior art indicated that several efforts have been made in the past to develop methods and systems to automate electronic health record documentation. However, these methods (prior arts) are not efficient in terms of seamless integration of machine learning engines to imitate the thought process of a Physician, expert coder and a scribe and automatically generate electronic health records in the EHR software.

Therefore, in light of foregoing discussion, there exists a need to automate the physician's clinical documentation process. Such automation requires structured medical data and in the clinical settings it is in the form of conversations between the doctor and the patient.

The present invention describes a method and a system for automatic electronic health record documentation.

### OBJECT OF THE INVENTION

The primary object of the present invention is to provide a method and a system for automatic electronic health record documentation;

Further object of the present invention is to provide to a method of training of the machine learning engines to imitate the thought process of a Physician, expert coder and a scribe and automatically generate electronic health records in the EHR (Electronic Health Record) software.

### SUMMARY OF THE INVENTION

Embodiments of the present disclosure present technological improvements as solution to one or more of the above-mentioned technical problems recognized by the inventor in conventional practices and existing state of the art.

The present disclosure seeks to provide a method and a system for automatic electronic health record documentation.

In accordance to an aspect of the present invention, present invention is a knowledge engineered auto scribe for listening to the conversation between the patient and doctor and entering the data into the desired electronic health record.

According to further aspect of the present invention, the system automatic electronic health record documentation comprises non-transitory storage medium; a set of executable software instructions comprising: annotator module having a first sub-module programmed to tag words or phrases from the interactions with semantic concepts, and a second sub-module programmed to associate one or more of the semantic concepts with the interactions; and a bucket classification module that maps the relationships to one or more narrative sub-sections.

According to further aspect of the present invention, the method automatic electronic health record documentation comprises receiving narrative content; scanning the narrative content using a natural language processing engine; extracting relevant medical information from the narrative content; identifying the missing categories in the partly structured text and generating a structured response; and sectioning the structured response to the medical text for the various type of clinical comments and documenting it into a Soap Note format which is signed by the medical practitioner after review.

The objects and the advantages of the invention are achieved by the process elaborated in the present disclosure.

### BRIEF DESCRIPTION OF DRAWINGS

The accompanying drawings constitute a part of this specification and illustrate one or more embodiments of the invention. Preferred embodiments of the invention are described in the following with reference to the drawings, which are for the purpose of illustrating the present preferred embodiments of the invention and not for the purpose of limiting the same.

For simplicity and clarity of illustration, the drawing figures illustrate the general manner of construction, and descriptions and details of well-known features and techniques may be omitted to avoid unnecessarily obscuring the invention. Additionally, elements in the drawing figures are not necessarily drawn to scale. For example, the dimensions of some of the elements in the figures may be exaggerated relative to other elements to help improve understanding of embodiments of the present invention. The same reference numerals in different figures denotes the same elements.

### IN THE DRAWINGS

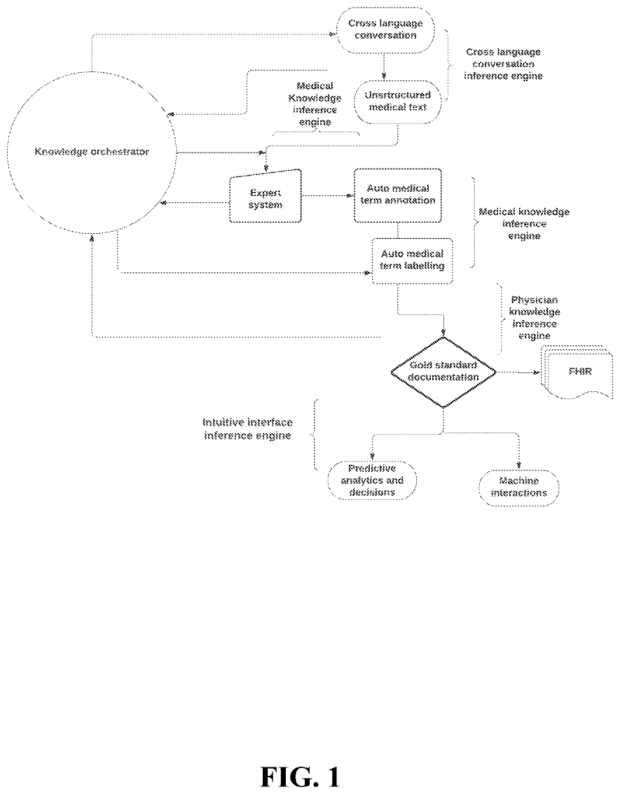

FIG. 1 illustrates the architecture of method and a system for automatic electronic health record documentation in accordance to the embodiment of the present invention;

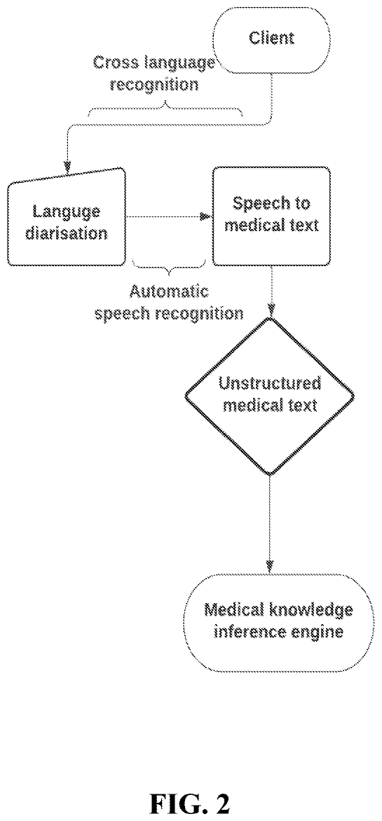

FIG. 2 illustrates the schematic diagram of a cross language conversation inference engine in accordance to the embodiment of the present invention;

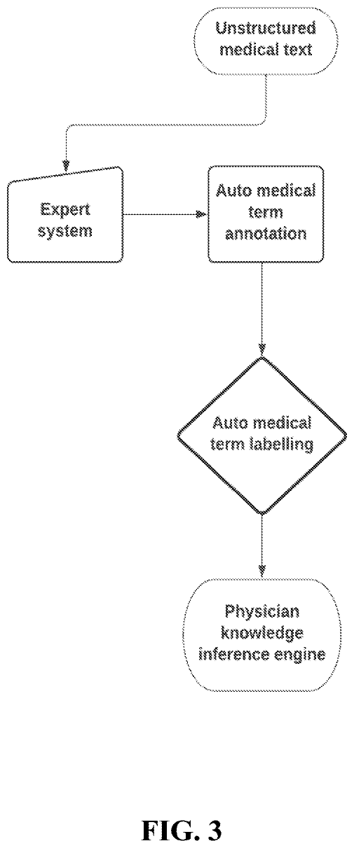

FIG. 3 illustrates the schematic diagram of a medical knowledge inference engine in accordance to the embodiments of the present invention;

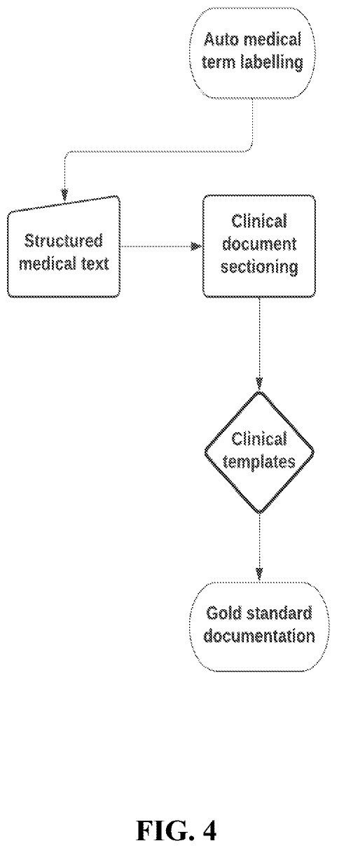

FIG. 4 illustrates the schematic diagram of physician knowledge inference engine in accordance to the embodiments of the present invention;

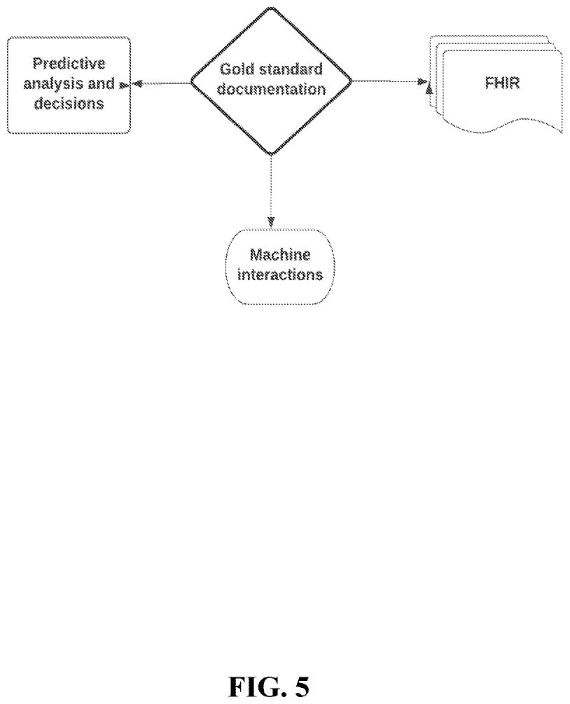

FIG. 5 illustrates the schematic diagram of intuitive interface inference engine in accordance to the embodiments of the present invention.

### DETAILED DESCRIPTION OF THE INVENTION

The following detailed description illustrates embodiments of the present disclosure and ways in which the disclosed embodiments can be implemented. Although some modes of carrying out the present disclosure have been disclosed, those skilled in the art would recognize that other embodiments for carrying out or practicing the present disclosure are also possible.

The present invention describes a method and a system for automatic electronic health record documentation.

Typical prior arts lack in the ability to acquire structured data during the conversation between the physician and patient to automate the volume of EHR (Electronic Health Record) documents.

The embodiments of the present invention use knowledge engineering and artificial intelligence to create auto machine learning component which will be in the physician's computer that listens to the conversation between doctor and the patient, understands the context, creates the document in SOAP (Subjective, Objective, Assessment and Plan) format and completes the charting process in the EHR software without the doctor entering any data.

This complete process needs no human intervention nor integration to the EHR software to complete the entire clinical documentation. In about a minute, the doctor will be able to have a note, an encounter form, a prescription, a lab sheet, and/or a pathology sheet. The doctor can just focus on achieving the best outcomes for the patients. No additional burden on documentation so the chances of burnout due to documentation is alleviated. The doctor can see more patients and improve his productivity too.

The embodiments of the present invention relate to knowledge engineering systems, and methods for creating auto machine learning system that will automatically generate meaningful documents from patient-Physician conversation.

The knowledge engineered auto machine learning system includes mimicking how the particular Physician will summarize his/her conversation with patient into a documented format and training the Artificial Intelligence (AI) module to execute the unique workflow of each Physician. It includes deciphering what the doctor and patient is saying in a conversation, breaking down the passage into chunks and extracting medically relevant text from the passage.

According to the embodiments of the present invention, the medically relevant text is mapped to different ontologies and grouped into a document under different subsections with respective codes (ICD, CPT, SNOMED, RXNORM) in a matter of minutes. The document after physician's approval is entered into the physician's EHR software without integration.

The present invention is a series of auto machine learning engines that infer based on descriptive medical knowledge and procedural Physician knowledge using multiple knowledge engineered auto machine learning models.

Structured data is the bed rock to automate any machine learning process. There exist auto machine learning models in healthcare in diagnostics, imaging and other areas where there is structured medical data. However, there has been none to auto ML the clinical documentation process as there is no technology available to convert the patient doctor conversation to structured medical data.

The transformation of conversation to structured clinical electronic health record process goes through four main elements. The steps are broken down to achieve the correct medical data for the auto machine learning to mimic the physician electronic health record documentation. The four interdependent elements of the present invention are as below.
1. Cross Lingual Conversation Inference Engine:
   This engine handles the cross lingual diarisation and cross language to unstructured medical text conversion.
2. Medical knowledge Inference Engine:
   This engine does auto subject diarisation with possible medical causes, medical featurization and medical data labelling.
3. Physician Knowledge Inference Engine:
   This engine has the trained models for various clinical notes that is used in clinical documentation.
4. Intuitive Frontend Inference Engine:
   Server less interface to humans and other software systems like EHR and FHIR

Each of the elements has various methods at its disposal to select the one that provides the most reliable outcome. Though there are series of auto ML's the evaluation methods used are very light in terms of their required computational power. Hence, the performance is very high.

1. Cross-Language Conversation Inference Engine

Cross language conversation inference is the process to perform language segmentation and recognition, in a code-switched speech between the physician and patient. The present invention uses conversational code-switch corpus. It has been programmed to handle frequent code-switching so that the average language interval may be as short as few seconds. Language diarisation itself is a challenging task, when processing such short segments, the complication becomes manifold. A language diarisation system is built using knowledge engineering to set the medical context feature. This is a top-down approach to medical data extraction. This prepares the unstructured medical text content for the next medical data identification element.

Since the response time and online processing are the crucial factors in clinical settings the cross-language speaker diarisation engine is directly integrated into the Automatic Speech Recognition (ASR) pipeline, while the medical featurization is happening in parallel. Highlights of this engine are:
Online speaker cross language diarisation machine learning uses neural network-based cross language diarisation which gives real-time and low latency aspect in language diarisation;
Tightly integrated speaker diarisation and ASR auto machine learning, improve the accuracy of speaker diarisation. The joint modelling approach leverages the inter-dependency between speaker diarisation and ASR to better perform both tasks;
Diarisation and ASR use a medical featurization machine learning model in parallel that is trained on medical data to provide excellent result due to domain match;
CLIE (Cross Lingual Inference engine) can handle cross language conversations of up to 15 leading languages of the world including English, Latin, Arabic, French, Germany Spanish, Malaysian Bahasa, Indonesian Bahasa, Hindi, Tamil, Telugu, Kannada and Malayalam.

One of the major factors in the success of the invention is because of the effectiveness in capturing the medical features in conversation as unstructured medical text by the Cross Lingual Inference engine. The production data is looped back to this engine to train the auto machine learning for constant improvement of accuracy at this stage. This unstructured medical text is the input to the Medical Knowledge Inference Engine (MKIE).

2. Medical Knowledge Inference Engine

MKIE is the auto machine learning tool to prepare the unstructured medical conversation text with medical labels to be processed by PKIE, which prepares the final clinical documentation in the form electronic health records in the EHR software. The auto annotator ML models provide the medical possibilities in the unstructured medical text like how the physicians mind infers, the medical expert auto ML models derive the structured medical data, like how the medical experts would look in the possibilities and the labelling auto ML streamlines the labels as per the global medical coding standards for these features and prepares the structured medical data for the PKIE element to generate the electronic health record.

MKIE is trained to understand specific medical information. Training data is categorised and annotated for the specific use in clinical documentation and electronic health records. With high-quality, human-powered expert medical data models and annotation models MKIE delivers excellent structured data results. The labelling rules engine ends up with a structured medical data pool for the encounter for PKIE to process further. MKIE has three auto ML engines.

Auto Medical term annotation—A domain-specific pre-trained medical model using un-labelled medical texts. This is constantly trained to ensure that the system is able to identify newer possibilities. Like how new possibilities unfolded at break neck speed during the COVID pandemic.

Auto Medical Expert—This is specialised and explicit medical knowledge of facts and procedures. This is created using medical libraries, medical standards and various medical resources by the experts. Medical knowledge is in the form of facts and rules. This has a data acquisition module to keep it up to date with latest medical libraries and medical entities.

Auto Medical term labelling—This is a rule engine-based labelling mechanism for un-labelled medical texts to automatically annotate texts and create medical coding labels by collecting medical entities from the result processed by the two other auto ML's in MKIE.

This sets a valid medial data base—Right data type, right range of values, right data validity and consistency for PKIE to generate gold standard documentation. No medical information is lost in the conversation. Everything is captured and is in the form of medical data.

3. Physician Knowledge Inference Engine

PKIE Auto ML is built using neural networks and deep learning of clinical documentation process. This auto ML is trained to deliver gold standard clinical documentation.

PKIE handle the following clinical documentation including FHIR interoperability:
Consultation note
Discharge summary
History and physical note
Procedural note
Progress note
Imaging narrative
Laboratory report
Pathology report

This model is trained on procedure of clinical documentation in Gold standards. Clinical documentation procedural knowledge is written as rules for mapping sectioning from features in the labeled data relating to SOAP documentation.

4. Intuitive Interface Inference Engine

The Intuitive Interface Engine (IIIE) is a server less client that can react quickly to any changes in the client. It can open two-way interactive communication with the cloud server. IIIE sends request to a server and receive event-driven responses without having to poll the server for a reply IIIE machine learning facilities automation, predictive analytics, recognitions, machine interactions and medical data interoperability. The doctor will know how to use it on sight. No training needed.

The IIIE auto machine learning interface having very few parameters makes it simple for physicians to work with. The IIIE auto machine learning models are trained to deliver results as per the context set by physicians. It could be new encounter or follow up, the documentation is derived for the right context.

The front end has following key functions:
1. Simplifying the model output to what the physician wants by choosing the type of automation by understanding the and make the whole back end as a black box. The automation for SOAP notes, ICD10, CPT, RX Norm and FHIR is available for new patients and follow up patients based on this context of the encounter.
2. Provide clinical documentation decision support through prediction based medical domain specific logic. Drug interaction, document completeness level and patient education content are auto suggested to the physician.
3. To send the final clinical documentation to EHR, FHIR or other modes through computer vision.
4. To train, test and monitor the personalized models for each physician based on the EHR software and the documentation process.
5. The front end also provides means for physician to intervene when ever needed and finally authorize the electronic health record before sign-off.

The logistics of datafication of the physician encounter process is very complex requiring heterogeneous knowledge. Using knowledge engineering concepts to model the pattern and generate data in a consistent and reliable way through out in multiple waves has resulted in this invention that would change the way the healthcare industry operates. Successful datafication of the clinical practice will lead large scale digitalization and personalization in the future.

### Advantages Offered by the Proposed Invention are Summarized Below

Cost advantage—No investment on the hardware or human scribe, it is completely automated and cloud enabled.
Time saving—Time is the most valuable asset for doctors. This invention automates the complete clinical documentation process. In about a minute, the doctor will be able to have a note, an encounter form, a prescription, a lab sheet, and/or a pathology sheet by not typing even a single letter.
Zero integration: Integration is complicated. When two systems try to talk to each other in real-time, data integrity and data corrections are a huge challenge. Instead of trying to attain real-time integration we have used the computer vision model for integration which mimics a human being entering data with all validation and controls.
No Data entry: The doctor and patients conversation is intelligently translated to medical documentation context and then automatically translated to the target EMR with no data entry by the doctor or scribe.
Intuitive system: The system needs no separate implementation process or cost. The doctor just has to download and start using it. The doctor will know how to use it on sight.
Cross lingual: Most of the patients and doctors will be able to converse in their language of preference, the system will be able to understand the languages of the target population
Better user experience: Multi form factor including desktop, tab or mobile irrespective of the EHR form factor.
Gold standard documentation: Even if the EMR has lesser capability, the present invention can deliver gold standard documentation to meet ONC's level 1 to 3 certification mandate.

According to an embodiment of the present invention, the present invention is a knowledge engineered auto scribe for listening to the conversation between the patient and doctor and entering the data into the desired EHR.

The present invention transcribes the conversation between the patient and doctor. The machine learning model or the system has been trained at the voice level to identify medical concepts spoken in different accent. This model is able to auto identify the medical context in speech. The patterns for different accent are trained as models using medical concept and algorithms.

After transcribing the conversation, the present invention infers medically relevant data from the unstructured text passage by passing through a plurality of databases. The unstructured voice text passes through ML's that validate medical text patterns, spell checking, split and merge corrector models for formatting data accurately in the language spoken. This generates an unstructured medical text.

According to exemplary embodiments of the present invention, the automated system uses knowledge engineering to convert recordings or diarized texts of interactions between a first person and a second person into a narrative, comprising: a non-transitory storage medium; a set of executable software instructions comprising: (i) annotator module having a first sub-module programmed to tag words or phrases from the interactions with semantic concepts, and a second sub-module programmed to associate one or more of the semantic concepts with the interactions; and (ii) a bucket classification module that maps the relationships to one or more narrative sub-sections. The models are trained to expect a sequence of likely speech flow of the doctor and patient. It mainly deals with finding a structure or pattern in a collection of uncategorized data. This translates and generates desired language output in partly structured medical text.

The automated system identifies the subject and the intent (the patients need/chief complaint) of the subject and extracts medical concepts associated with intent. Based on the intent the appropriate clinical pathway model that have been trained to look for specific words in the chucked concepts and groups the broken-down chunks i.e., bucket to medically relevant ontologies and sub-sections. Specialty based NLP medical concepts algorithm looks at the un categorized data and maps to medical concepts from using a specialty NLP ML where the data is chunked under the concepts of chief complaints, past history, HPI, diagnosis, treatment, medication and diagnostics are auto identified. Unidentified words/sentences are highlighted.

The supervised medical expert ML identifies the missing categories provides a structured response comprised of chief complaint, history of presenting illness, History (medication history, social history, family history, diet and exercise), vitals, review of systems, physical examination, diagnosis, procedure, lab tests, prescription and follow up. This model identifies the missing categories in the partly structured text using the patient encounter protocol. The document is now in a structured medical terms state.

The auto medical coding machine learning uses the structure medical terms, context and the treatment protocol to identify the right medical codes. The response also comprised of medical codes of ICD, CPT, Rx Norms and Snowmed CT codes.

According to further embodiments of the present invention, the response is sectioned to the medical text for the various type of clinical comments like discharge summary, soap notes, progress notes as per the physician's way of doing and is put into a documented format (SOAP NOTE). After reviewing the response in the SOAP format, the doctor signs the document.

This ML has been trained on the right clinical documentation standards for various type of clinical pathways. This matches patterns to verify the document and suggest improvements to meet gold standards.

The knowledge engineered auto ML which is built using the skill builder for every physician. This learns the EHR workflow of the physician and mimics the clinical workflow and protocol of the particular doctor to ensure all elements as per the doctors practice is in place and it logs into the EHR and enter the data as the physician will do.

We claim:

1. A system for automatic electronic health record documentation, the said system comprising:
a non-transitory storage medium;
a set of executable software instructions comprising:
a) annotator module having a first sub-module programmed to tag words or phrases from the interactions with semantic concepts, and a second sub-module programmed to associate one or more of the semantic concepts with the interactions; and
b) a bucket classification module that maps the relationships to one or more narrative sub-sections;
characterized by automatic identification of the medical context in recoding; use of knowledge engineering to convert recordings or diarized texts of interactions between a medical practitioner and a patient into a narrative; voice level trained machine learning model to identify medical concepts spoken in different accent.

2. The system as claimed in claim 1, wherein the annotator module identifies the subject and the intent of the subject and extracts medical concepts associated with intent.

3. The system as claimed in claim 1, wherein the bucket classification module identifies specific words in the chucked concepts and groups the broken-down chunks to medically relevant ontologies and sub-sections based on the intent.

4. The system as claimed in claim 1, wherein the natural language processing (NLP) algorithm maps the uncategorized data to medical concepts using a specialty NLP machine learning (ML) where the data is chunked under the concepts of chief complaints, past history, HPI, diagnosis, treatment, medication and diagnostics are auto identified and highlights the unidentified words and sentences.

5. The system as claimed in claim 1, wherein the supervised medical expert machine learning identifies the missing categories in the partly structured text using the patient encounter protocol and provides a structured response consisting of chief complaint; history of present illness including medication history, social history, family history, diet and exercise; vitals; review of systems; physical examination; diagnosis; procedure; lab tests; prescription; follow up and medical codes of ICD, CPT, Rx Norms and Snowmed CT codes.

6. The system as claimed in claim 1, wherein the structured response is sectioned to the medical text for the various type of clinical comments like discharge summary, soap notes, progress notes as per the physician's way of doing and is put into a documented format (SOAP NOTE) which is signed by the medical practitioner after review.

7. The system as claimed in claim 1, wherein the patterns for different accent are trained as models using medical concept and algorithms.

8. A method for automatic electronic health record documentation, the said method comprising:
receiving narrative content;
scanning the narrative content using a natural language processing engine to identify the subject and the intent of the subject and extracts medical concepts associated with intent;
extracting information from the narrative content including the concepts of chief complaints, past history, HPI, diagnosis, treatment, medication and diagnostics are auto identified and highlights the unidentified words and sentences; and
identifying the missing categories in the partly structured text and generating a structured response
sectioning the structured response to the medical text for the various type of clinical comments like discharge summary, soap notes, progress notes as per the physician's way of doing and documenting it into a Soap Note format which is signed by the medical practitioner after review.

9. The method as claimed in claim 8, wherein the transcribed unstructured text is passed through a plurality of databases to infer medially relevant data.

10. The method as claimed in claim 8, wherein the unstructured voice text passes through machine learning that validates the medical text patterns, spell checking, split and merge corrector models for formatting data accurately in the language spoken generating an unstructured medical text.
